(19) United States
(12) Patent Application Publication
(10) Pub. No.: US 2008/0033538 A1
(43) Pub. Date: Feb. 7, 2008

(54) IMPLANT MADE OF A BIOCORRODIBLE METALLIC MATERIAL HAVING A COATING MADE OF AN ORGANOSILICON COMPOUND

(75) Inventors: Alexander Borck, Aurachtal (DE); Alexander Rzany, Nuernberg (DE); Eric Wittchow, Nuernberg (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(73) Assignee: BIOTRONIK VI PATENT AG, Baar (CH)

(21) Appl. No.: 11/832,186

(22) Filed: Aug. 1, 2007

(30) Foreign Application Priority Data
Aug. 7, 2006 (DE) 10 2006 038 231.5

(51) Int. Cl. A61F 2/82; A61L 33/00

(52) U.S. Cl. 623/1.46; 427/2.24

(57) ABSTRACT

An implant made of a biocorrodible metallic material having a coating made of an organosilicon compound.

### PRIORITY CLAIM

This patent application claims priority to German Patent Application No. 10 2006 038 231.5, filed Aug. 7, 2006, the disclosure of which is incorporated herein by reference in its entirety.

### FIELD

The present disclosure relates to an implant made of a biocorrodible metallic material having a coating made of a silicon compound as well as an associated method for producing the implant.

### BACKGROUND

Medical implants of greatly varying intended purposes are known in the art. Frequently, only temporary residence of the implant in the body is required to fulfill the medical purpose. Implants made of permanent materials, i.e., materials which are not degraded in the body, are to be removed again, because rejection reactions of the body may occur in the medium and long term even in the event of high biocompatibility.

One approach for avoiding a further surgical intervention comprises molding the implant entirely or partially from a biocorrodible material. For purposes of the present disclosure, biocorrosion is microbial procedures or processes caused solely by the presence of bodily media, which result in a gradual degradation of the structure comprising the material. At a specific time, the implant, or at least the part of the implant which comprises the biocorrodible material, loses its mechanical integrity. The degradation products are largely resorbed by the body. These products, such as magnesium, for example, may even provide a local therapeutic effect. Small quantities of alloy components which may not be resorbed are tolerable.

Biocorrodible materials have been developed, inter alia, on the basis of polymers of synthetic nature or natural origin. The mechanical material properties (low plasticity), but also the sometimes low biocompatibility of the degradation products of the polymers (partially increased thrombogenicity, increased inflammation), limit the use significantly, however. Thus, for example, orthopedic implants frequently must withstand high mechanical strains; and vascular implants, such as stents, must meet very special requirements for modulus of elasticity, brittleness, and moldability depending on design.

One promising approach for solving the problem provides the use of biocorrodible metal alloys. Thus, it is suggested in German Patent Application No. 197 31 021 A1 that medical implants be molded from a metallic material whose main component is selected from the group consisting of alkali metals, alkaline earth metals, iron, zinc, aluminum, combinations thereof and the like. Alloys based on magnesium, iron, zinc and the like are described as especially suitable. Secondary components of the alloys may be manganese, cobalt, nickel, chromium, copper, cadmium, lead, tin, thorium, zirconium, silver, gold, palladium, platinum, silicon, calcium, lithium, aluminum, zinc, iron, combination thereof and the like. Furthermore, the use of a biocorrodible magnesium alloy having a proportion of magnesium greater than 90%, yttrium 3.7-5.5%, rare earth metals 1.5-4.4%, and the remainder less than 1% is known from German Patent Application No. 102 53 634 A1, which is suitable, in particular, for producing an endoprosthesis, e.g., in the form of a self-expanding or balloon-expandable stent. Notwithstanding the progress achieved in the field of biocorrodible metal alloys, the alloys known up to this point are also only capable of restricted use because of their material properties, such as strength and corrosion behavior, for example. The relatively rapid biocorrosion of magnesium alloys, in particular, in the field of structures which are strongly mechanically loaded, limits their use.

Both the foundations of magnesium corrosion and also a large number of technical methods for improving the corrosion behavior (in the meaning of reinforcing the corrosion protection) are known in the art. It is known, for example, that the addition of yttrium and/or further rare earth metals to a magnesium alloy provides a slightly increased corrosion resistance in seawater.

One approach provides generating a corrosion-protecting layer on the molded body comprising magnesium or a magnesium alloy. Known methods for generating a corrosion-protecting layer have been developed and optimized from the viewpoint of technical use of the molded body, but not a medical-technical use in biocorrodible implants in a physiological environment. These known methods comprise, for example, application of polymers or inorganic cover layers, production of an enamel, chemical conversion of the surface, hot gas oxidation, anodization, plasma spraying, laser beam remelting, PVD methods, ion implantation, or lacquering.

Typical technical areas of use of molded bodies made of magnesium alloys outside medical technology normally require extensive suppression of corrosive processes. Accordingly, the goal of most technical methods is complete inhibition of corrosive processes. In contrast, the goal for improving the corrosion behavior of biocorrodible magnesium alloys is not complete suppression, but rather only inhibition of corrosive processes. For this reason alone, most known methods for generating a corrosion protection layer are not suitable. Furthermore, toxicological aspects must also be taken into consideration for a medical-technical use. Moreover, corrosive processes are strongly dependent on the medium in which they occur, and, therefore, unrestricted transfer of the findings for corrosion protection obtained under typical environmental conditions in the technical field to the processes in a physiological environment is not possible. Finally, in multiple medical implants, the mechanisms on which the corrosion is based may also deviate from typical technical applications of the material. Thus, for example, stents, surgical suture material, or clips are mechanically deformed in use, so that the partial process of tension cracking corrosion may have great significance in the degradation of these molded bodies.

In addition, it is to be noted that in implants such as stents, local high plastic deformations of the main body occur. Conventional methods such as generating a dense magnesium oxide layer, which may also contain OH groups, are not expedient for this application. The ceramic properties of the cover layer would result in local chipping and/or cracking. The corrosion would thus be locally focused in an uncontrolled way in the area of the mechanically loaded points, which are actually particularly to be protected.

German Patent Application No. 101 63 106 A1 provides changing the magnesium material in its corrosivity by modification with halogenides. The magnesium material is to be used for producing medical implants. The halogenide is preferably a fluoride. The material is modified by alloying halogen compounds in salt form. The composition of the magnesium alloy is accordingly changed by adding the halogenides to reduce the corrosion rate. Accordingly, the entire molded body comprising such a modified alloy will have an altered corrosion behavior. However, further material properties, which are significant in processing or also affect the mechanical properties of the molded body resulting from the material, may be influenced by the alloying.

Furthermore, coatings for implants made of non-biocorrodible, i.e., permanent materials, are known, which are based on organosilicon compounds. Thus, for example, German Patent Application No. 699 12 951 T2 describes an intermediate layer made of a functionalized silicone polymer, such as siloxanes or polysilanes. U.S. Patent Publication No. 2004/0236399 A1 discloses a stent having a silane layer, which is covered by a further layer.

### SUMMARY

The present disclosure provides an alternative or improved coating for implants made of a biocorrodible material, which cause a temporary inhibition, but not complete suppression, of the corrosion of the material in a physiological environment.

The present disclosure provides several exemplary embodiments of the present invention, some of which are discussed below.

One aspect of the present disclosure provides an implant made of a biocorrodible metallic material having a coating made of an organosilicon compound of formula (1):

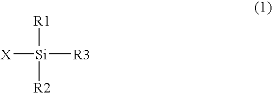

with X signifying a O, S, or N functionality on a surface of the implant, via which a covalent bond of the organosilicon compound to the surface of the implant occurs; R1 and R2, established independently of one another, being a substituted or unsubstituted alkyl residue having 1 to 5 C atoms or an oxygen bridge to a neighboring organosilicon compound; and R3 being a substituted or unsubstituted alkyl residue or an alkyl bridge to a neighboring organosilicon compound and the alkyl residue/alkyl bridge having 3 to 30 C atoms, 1, 2, or 3 C atoms being replaceable by a heteroatom selected from the group O, S, and N.

Another aspect of the present disclosure provides a method for producing an implant made of a biocorrodible metallic material having a coating made of an organosilicon compound of formula (1):

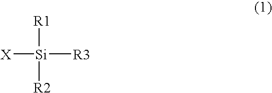

with X signifying a O, S, or N functionality on a surface of the implant, via which a covalent bond of the organosilicon compound to the surface of the implant occurs; R1 and R2, established independently of one another, being a substituted or unsubstituted alkyl residue having 1 to 5 C atoms or an oxygen bridge to a neighboring organosilicon compound; and R3 being a substituted or unsubstituted alkyl residue or an alkyl bridge to a neighboring organosilicon compound and the alkyl residue/alkyl bridge having 3 to 30 C atoms, 1, 2, or 3 C atoms being replaceable by a heteroatom selected from the group O, S, and N; and the method comprising the following steps: (a) providing a blank for the implant comprising the biocorrodible metallic material; (b) optionally, pretreating a blank surface to generate O, S, or N functionalities; and (c) coating the blank surface using an organosilicon reagent, which reacts between silicon and a O, S, or N functionality to form a covalent bond, either the organosilicon compound of formula (1) forming directly, or first a precursor organosilicon compound occurring, which is converted via further treatment steps into the organosilicon compound of formula (1).

It has been shown that the application of a coating of the cited composition does not result in the formation of a protective layer which completely or extensively inhibits the corrosion in a physiological environment. In other words, corrosion of the implant still occurs in a physiological environment, but at significantly reduced speed.

The biocorrodible metallic material is preferably a biocorrodible alloy selected from the group of elements consisting of magnesium, iron, and tungsten; in particular, the material is a biocorrodible magnesium alloy. For purposes of the present disclosure, an alloy is a metallic structure whose main component is magnesium, iron, or tungsten. The main component is the alloy component whose weight proportion in the alloy is highest. A proportion of the main component is preferably more than 50 weight-percent (wt.-%,), more preferably, more than 70 wt.-%.

If the material is a magnesium alloy, the material preferably contains yttrium and further rare earth metals, because an alloy of this type is distinguished due to the physiochemical properties and high biocompatibility, in particular, also the degradation products.

A magnesium alloy of the composition rare earth metals 5.2-9.9 wt.-%, thereof yttrium 3.7-5.5 wt.-%, and the remainder less than 1 wt.-% is especially preferable, magnesium making up the proportion of the alloy to 100 wt.-%. This magnesium alloy has already confirmed special suitability experimentally and in initial clinical trials, i.e., the magnesium alloy displays high biocompatibility, favorable processing properties, good mechanical characteristics, and corrosion behavior adequate for the intended uses. For purposes of the present disclosure, the collective term “rare earth metals” is understood to include scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70), lutetium (71), combinations thereof and the like.

The alloys of the elements magnesium, iron, or tungsten are to be selected in the composition in such a way that they are biocorrodible. For purposes of the present disclosure, alloys are biocorrodible in which degradation occurs in a physiological environment, which finally results in the entire implant or the part of the implant made of the material losing its mechanical integrity. Artificial plasma, as has been previously described according to EN ISO 10993-15:2000 for biocorrosion assays (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l), is used as a testing medium for testing the corrosion behavior of an alloy coming into consideration. For this purpose, a sample of the alloy to be assayed is stored in a closed sample container with a defined quantity of the testing medium at 37° C. At time intervals, tailored to the corrosion behavior to be expected, of a few hours up to multiple months, the sample is removed and examined for corrosion traces in a way known in the art. The artificial plasma according to EN ISO 10993-15:2000 corresponds to a medium similar to blood and thus represents a possibility for simulating a physiological environment reproducibly.

For purposes of the present disclosure, the term corrosion relates to the reaction of a metallic material with its environment, a measurable change to the material being caused, which, upon use of the material in a component, results in an impairment of the function of the component. For purposes of the present disclosure, a corrosion system comprises the corroding metallic material and a liquid corrosion medium, which simulates the conditions in a physiological environment in composition or is a physiological medium, particularly blood. On the material side, the corrosion factors influence the corrosion, such as the composition and pretreatment of the alloy, microscopic and submicroscopic inhomogeneities, boundary zone properties, temperature and mechanical tension state, and, in particular, the composition of a layer covering the surface. On the side of the medium, the corrosion process is influenced by conductivity, temperature, temperature gradients, acidity, volume-surface ratio, concentration difference, flow velocity, combinations thereof and the like.

Redox reactions occur at the phase boundary between material and medium. For a protective and/or inhibiting effect, existing protective layers and/or the products of the redox reactions must implement a sufficiently dense structure, have increased thermodynamic stability in relation to the environment, and have little solubility or be insoluble in the corrosion medium. In the phase boundary, more precisely in a double layer forming this area, adsorption and desorption processes occur. The procedures in the double layer are influenced by the cathodic, anodic, and chemical partial processes occurring there. In magnesium alloys, typically a gradual alkalinization of the double layer is to be observed. Foreign material deposits, contaminants, and corrosion products influence the corrosion process. The procedures during corrosion are highly complex and either cannot be predicted at all or can be predicted only to a limited extent precisely in connection with a physiological corrosion medium, i.e., blood or artificial plasma, because there is no comparative data. For this reason, finding a corrosion-inhibiting coating, i.e., a coating which only is used for temporary reduction of the corrosion rate of a metallic material of the composition cited above in a physiological environment, is a measure outside the routine of one skilled in the art. This is particularly true for stents, which are subjected to local high plastic deformations at the time of implantation. Conventional approaches using rigid corrosion-inhibiting layers are unsuitable for conditions of this type.

The procedure of corrosion may be quantified by specifying a corrosion rate. Rapid degradation is connected to a high corrosion rate, and vice versa. A surface modified in accordance with the present disclosure would result in reduction of the corrosion rate in regard to the degradation of the entire molded body. The corrosion-inhibiting coating may be degraded in the course of time and/or may only protect the areas of the implant covered thereby to a lesser and lesser extent. Therefore, the course of the corrosion rate is nonlinear for the entire implant. Rather, a relatively low corrosion rate results at the beginning of the occurring corrosive processes, which increases in the course of time. This behavior is understood as a temporary reduction of the corrosion rate and distinguishes the corrosion-inhibiting coating. In the case of coronary stents, the mechanical integrity of the structure is to be maintained over a period of time of three months after implantation.

For purposes of the present disclosure, implants are devices introduced into the body via a surgical method and comprise fasteners for bones, such as screws, plates, or nails, intestinal clamps, vascular clips, prostheses in the area of the hard and soft tissue, and anchoring elements for electrodes, in particular, of pacemakers or defibrillators. The implant entirely or partially comprises the biocorrodible material. If the implant only partially comprises the biocorrodible material, this part is to be coated accordingly.

The implant is preferably a stent. Stents of typical construction have a filigree structure made of metallic struts, which is first provided in a non-expanded state for introduction into the body and which is then expanded into an expanded state at the location of application. Special requirements exist for the corrosion-inhibiting layer in stents; the mechanical strain of the material during the expansion of the implant has an influence on the course of the corrosion process, and it is to be assumed that the tension crack corrosion will be greater in the strained areas. A corrosion-inhibiting layer takes this circumstance into consideration. Furthermore, a hard corrosion-inhibiting layer may chip off during the expansion of the stent and cracking in the layer during expansion of the implant may be unavoidable. Finally, the dimensions of the filigree of metallic structure are to be noted and, if possible, only a thin, but also uniform corrosion-inhibiting layer is to be generated. It has been shown that the application of the coating entirely or at least extensively meets these requirements.

The functionality on the surface of the implant necessary for binding the organosilicon compound of formula (1) may be provided, for example, by targeted pretreatment on the surface. Thus, a plasma treatment in oxygen-rich or nitrogen-rich atmosphere may precede the further steps in the production of the coating.

Residues R1 and R2 may carry further substituents, such as halogenides, particularly chlorine. However, the residues R1 and R2 are preferably unsubstituted and correspond to a substituent elected from the group consisting of methyl, ethyl, n-propyl, and i-propyl. If R1 or R2 is in oxygen bridge, the shared substituent binds two organosilicon compounds of formula (1) to one another. If R1 and R2 are each an oxygen bridge, a polymer network is formed from organosilicon compounds of formula (1).

R3 is a substituted or unsubstituted alkyl or heteroalkyl residue having 3 to 30 C atoms. For example, halogenides, particularly chlorine, aromatics, or heteroaromatic compounds may be provided as substituents. It is especially preferable if R3 carries a reactive substituent terminally, i.e., on the chain end facing away from the silicon. This reactive substituent may, for example, be an alcohol group, acid group, a vinyl compound, a urethane capped by isocyanate, an oxide, or an amine. By reaction with suitable substrates, the reactive substituent may be used for binding pharmaceutically active ingredients or biomolecules (e.g., oligonucleotides and enzymes), or for fixing further coatings (e.g., coupling to water-soluble carbodiimides).

Furthermore, R3 is preferably a substituted or unsubstituted alkyl residue having 5 to 15 C atoms, 1 to 3 C atoms being replaceable by a heteroatom, selected from the group consisting of O, N, and S. The substituent R3 is also preferably unbranched. The substituent may originate from the group of substituted or unsubstituted aromatic or heteroaromatic compounds, which are connected via a preferably unbranched alkyl chain of 1-5 carbon atoms to the silicon atom. Finally, R3 is preferably a residue selected from the group consisting of 3-mercapto-propyl, n-propyl, n-hexyl, n-octyl, n-decyl, n-tetradecyl, n-octadecyl, 3-aminopropyl, N-(2-aminoethyl)-3-aminopropyl, or N-(6-aminohexyl)-aminopropyl.

Preferably, R3 is a substituted or unsubstituted alkyl bridge to a neighboring organosilicon compound of formula (1) having 3 to 30 C atoms, 1, 2, or 3 C atoms being replaceable by a heteroatom selected from the group consisting of O, S, and N. This coating has an increased binding strength to the implant surface and resistance of the coating to hydrolysis. For preparation, preferably dipodal organosilicon reagents are used. Dipodal organosilicon compounds have two reactive silane groups connected to one another via an alkyl bridge, whose further residues allow a covalent bond to the implant surface on one hand and, on the other hand, correspond to the above-mentioned residues R1 and R2 or represent a precursor for producing these residues. Thus, the organosilicon reagent used for producing the coating may have alkoxy groups or halogenides, in particular chlorine, as leaving groups, which are used for covalent bonding or for introducing the residues R1 and R2. Suitable dipodal organosilicon reagents for producing the coating comprise, for example, bis-(triethoxysilyl)-ethane, 1,2-bis-(trimethoxysilyl)-decane, bis-(triethoxysilyl-propyl)-amine, and bis-[(3-trimethoxysilyl)propyl]-ethylendiamine. Mixtures of dipodal with monopodal silanes are preferably used for the coating. Typical mixture ratios are 1:5 to 1:10 (dipodal:monopodal).

A further aspect of the present disclosure relates to a method for producing an implant made of a biocorrodible metallic material, whose surface is covered by a coating made of an organosilicon compound of the above-mentioned type. The method comprises the following steps of (i) providing a blank for the implant made of the biocorrodible metallic material; (ii) optionally, pretreating a blank surface to generate O, S, or N functionalities; and (iii) coating the blank surface using an organosilicon reagent, which reacts between silicon and a O, S, or N functionality to form a covalent bond, either the organosilicon compound of formula (1) forming directly, or first a precursor organosilicon compound occurring, which is converted via further treatment steps into the organosilicon compound of formula (1).

Accordingly, the coatings may be generated from an organosilicon compound of formula (1) on the implant surface with the aid of the method.

In step (i) of the method, a blank for the implant is provided, e.g., in the form of a metallic main body for a stent.

In optional step (ii) of the method, the blank surface may be pretreated to establish the functionality necessary for the bonding of organosilicon compound on the surface of the implant. This may be performed, for example, by treatment using oxygen-rich or nitrogen-rich plasma, OH and NH functionalities resulting on the surface after the treatment. With corresponding reactive materials, OH groups may also be generated by immersion in water, bases, or acids.

In step (iii) of the method, the blank surface is coated using an organosilicon reagent. This work step comprises spraying the blank surface with the reagent or a solution of the reagent in a suitable solvent having a defined water content, for example.

The organosilicon reagent has a suitable leaving group, which is substituted while forming a covalent bond between silicon and one of the O, S, or N functionalities on the surface of the implant. The leaving group is preferably chlorine, a methoxy group, or an ethoxy group. Furthermore, the organosilicon reagent already either carries the identical residues R1 through R3 of the organosilicon compound of formula (1) to be produced, or the organosilicon reagent first only forms an intermediate stage, i.e., a precursor organosilicon compound results. The precursor organosilicon compound is then converted into the desired organosilicon compound of formula (1) by further treatment steps.

Examples of this exemplary embodiment via a precursor organosilicon compound particularly comprise organosilicon compounds of formula (1), in which R1 and/or R2 forms an oxygen bridge to a neighboring organosilicon compound (corresponding to a polysiloxane coating). The organosilicon reagent has, in addition to the residue R3, one or two leaving groups which later form the oxygen bridge of the residues R1 and/or R2. These leaving groups may comprise halogenides or a methoxy group, for example. After the bonding to the surface of the implant, cross-linking occurs in an aqueous alkaline environment to form the desired organosilicon compound of formula (1). Optionally, the workpiece may subsequently be neutralized within several hours by carbon dioxide in air.

### BRIEF DESCRIPTION OF THE DRAWINGS

The present disclosure is explained in greater detail in the following on the basis of exemplary embodiments and the associated drawings.

FIG. 1 shows a schematic representation to illustrate the procedures during coating of the implant surface;

FIG. 2 shows a schematic illustration of a coating, in which the organosilicon compound carries a reactive substituent terminally; and

FIG. 3 shows a schematic illustration of a coating in which the organosilicon compound is a polysiloxane.

### DETAILED DESCRIPTION

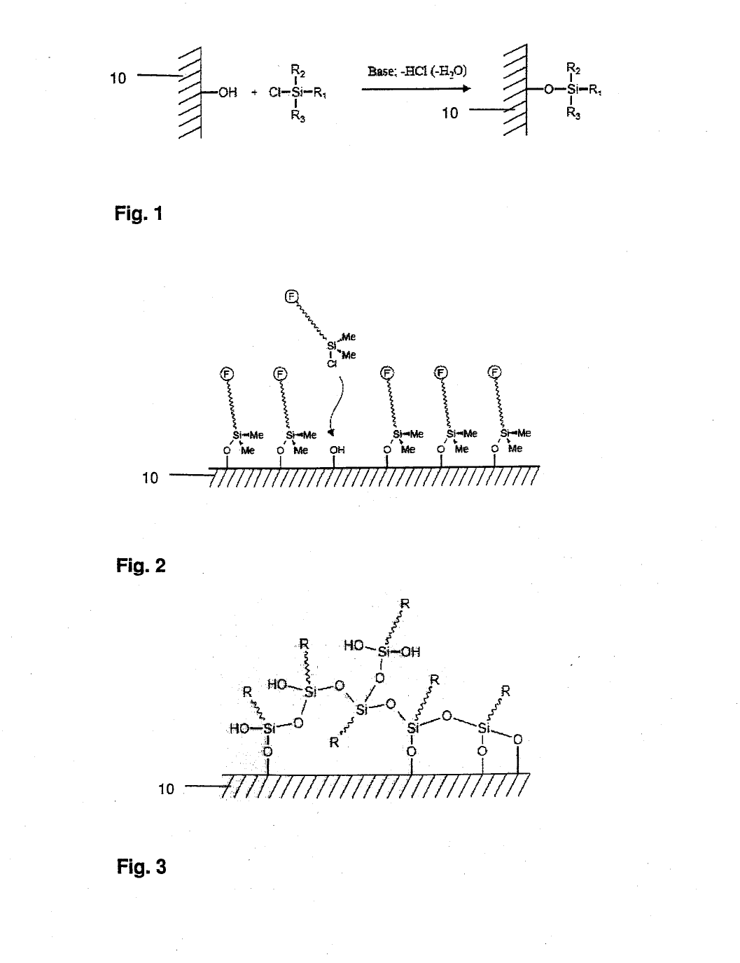

FIG. 1 is used for illustrating the procedures during coating of an implant surface 10 made of a biocorrodible metallic material. The implant surface 10 has a OH functionality. The OH functionality bonds covalently to the implant surface 10 by reaction with the chlorosilane shown under water-free basic conditions. The residues R1 through R3 of the chlorosilane are established as previously noted.

FIG. 2 schematically illustrates the sequences during functionalization of the implant surface 10 using a silane, which, in addition to two methyl groups, has a long-chain, unbranched alkyl residue having a terminally situated reactive group (identified by F). The long-chain residue forms a hydrophobic barrier layer. Due to the long-chain alkyl residues, which form a homogeneous, dense layer, the function as a corrosion-inhibiting barrier layer is maintained even in areas of high mechanical deformation of the main body. The organosilicon layer adapts itself to the given steric boundary conditions, a closed layer being maintained by the strong hydrophobic force on the alkyl residues situated in parallel.

FIG. 3 shows a coating made of a covalently bonded polysiloxane.

### EXAMPLES

### Example 1

### Stent Coating Using 3-aminopropyltriethoxysilane

Stents made of the biocorrodible magnesium alloy WE43 (93 wt.-% magnesium, 4 wt.-% yttrium (W), and 3 wt.-% rare earth metals (E) except for yttrium) were washed under ultrasound using isopropanol and dried.

A coating solution made of 18 ml water-free toluene, 2.2 ml aminopropyltriethoxysilane, and 1 ml triethylamine was used.

The stents were incubated for 4 hours at 75° C. in the coating solution, removed again, washed with toluene, and dried at approximately 90° C. for an hour in the vacuum furnace.

### Example 2

### Stent Coating Using 3-mercapto-propyl-trimethoxysilane

Stents made of the biocorrodible magnesium alloy WE43 (93 wt.-% magnesium, 4 wt.-% yttrium (W), and 3 wt.-% rare earth metals (E) except for yttrium) were washed using chloroform and dried.

A coating solution made of 90 wt.-% methanol, 6 wt.-% water, and 4 wt.-% 3-mercapto-propyl-trimethoxysilane (PropS-SH) was used. The pH value was adjusted to 4.5-5.5 by adding acetic acid

The stents were immersed at room temperature in the coating solution for 30 minutes, removed again, washed using methanol, and dried at approximately 60° C. for one hour in the vacuum furnace.

### Example 3

### Stent Coating Using n-octadecyltrichlorosilane

Stents made of the biocorrodible magnesium alloy WE43 (93 wt.-% magnesium, 4 wt.-% yttrium (W), and 3 wt.-% rare earth metals (E) except for yttrium) were washed using chloroform and dried.

A coating solution made of 95 wt.-% chlorobenzene and 5 wt.-% n-octadecyltrichlorsilane was used.

The stents were immersed under dried nitrogen for 5 minutes at room temperature in the coating solution. After the silanization, the stents were washed using chlorobenzene, cleaned for 10 minutes in ethanol under ultrasound, and dried at approximately 60° C. for one hour in the vacuum furnace.

All patents, patent applications and publications are incorporated by reference herein in their entirety.

What is claimed:

1. An implant made of a biocorrodible metallic material having a coating made of an organosilicon compound of formula (1): 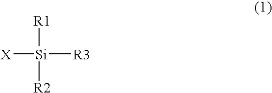
X signifying a O, S, or N functionality on a surface of the implant, via which a covalent bond of the organosilicon compound to the surface of the implant occurs;
R1 and R2, established independently of one another, being a substituted or unsubstituted alkyl residue having 1 to 5 C atoms or an oxygen bridge to a neighboring organosilicon compound; and
R3 being a substituted or unsubstituted alkyl residue or an alkyl bridge to a neighboring organosilicon compound and the alkyl residue/alkyl bridge having 3 to 30 C atoms, 1, 2, or 3 C atoms being replaceable by a heteroatom selected from the group O, S, and N.

2. The implant of claim 1, wherein the biocorrodible metallic material is a biocorrodible alloy selected from the group consisting of magnesium, iron, and tungsten.

3. The implant of claim 2, wherein the biocorrodible metallic material is a magnesium alloy

4. The implant of claim 1, wherein the implant is a stent.

5. The implant of claim 1, wherein R1 and R2, established independently of one another, correspond to a substituent selected from the group consisting of methyl, ethyl, n-propyl, and i-propyl.

6. The implant of claim 1, wherein R1 and R2, established independently of one another, are a substituted or unsubstituted alkyl residue having 1 to 5 C atoms.

7. The implant of claim 1, wherein R3 is a substituted or unsubstituted alkyl residue having 5 to 15 C atoms, 1 to 3 C atoms being replaceable by a heteroatom selected from the group consisting of O, N, and S.

8. The implant of claim 1, wherein R3 carries a reactive substituent terminally.

9. A method for producing an implant made of a biocorrodible metallic material having a coating made of an organosilicon compound of formula (1): 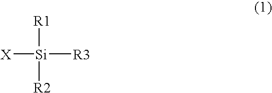
X signifying a O, S, or N functionality on a surface of the implant, via which a covalent bond of the organosilicon compound to the surface of the implant occurs;
R1 and R2, established independently of one another, being a substituted or unsubstituted alkyl residue having 1 to 5 C atoms or an oxygen bridge to a neighboring organosilicon compound; and
R3 being a substituted or unsubstituted alkyl residue or an alkyl bridge to a neighboring organosilicon compound and the alkyl residue/alkyl bridge having 3 to 30 C atoms, 1, 2, or 3 C atoms being replaceable by a heteroatom selected from the group O, S, and N;
and the method comprising the following steps:
(a) providing a blank for the implant comprising the biocorrodible metallic material;
(b) optionally, pretreating a blank surface to generate O, S, or N functionalities; and
(c) coating the blank surface using an organosilicon reagent, which reacts between silicon and a O, S, or N functionality to form a covalent bond, either the organosilicon compound of formula (1) forming directly, or first a precursor organosilicon compound occurring, which is converted via further treatment steps into the organosilicon compound of formula (1).
